(19) United States
(12) Patent Application Publication
(10) Pub. No.: US 2007/0038177 A1
(43) Pub. Date: Feb. 15, 2007

(54) Self securing chest tubes and methods

(76) Inventors: Anil K. Sinha, Lake Jackson, TX (US); Daniel DeOliveira, Lake Jackson, TX (US)

Correspondence Address: OSHA LIANG L.L.P., 1221 MCKINNEY STREET, SUITE 2800, HOUSTON, TX, 77010, US

(21) Appl. No.: 11/503,727

(22) Filed: Aug. 14, 2006

Related U.S. Application Data
(60) Provisional application No. 60/707,708, filed on Aug. 12, 2005.

(51) Int. Cl. A61M 29/00

(52) U.S. Cl. 604/96.01; 604/101.01

(57) ABSTRACT

A chest tube includes a hollow tube having a first end and a second end; a first securing device disposed proximate the first end; and a mechanism to activate the first securing device. A method for draining a pleural cavity with a chest tube includes the steps of: (1) inserting a chest tube into the pleural cavity, wherein the chest tube comprising a hollow tube comprising: a first end; and a second end; and a first securing device disposed proximate the first end; and (2) activating the first securing device.

### CROSS REFERENCE TO RELATED APPLICATIONS

The application claims the benefits of Provisional Application Ser. No. 60/707,708, filed on Aug. 12, 2005, entitled “Self Secure Chest Tube,” which is incorporated by reference in its entirety. In addition, this application is related to the following two applications filed on the same date: “Chest Tube Air Counters and Methods,” and “Apparatus and Methods for Safe and Efficient Placement of Chest Tubes.” These two related applications are also incorporated by reference in their entireties.

### BACKGROUND OF INVENTION

1. Field of the Invention

The invention relates generally to chest tubes used in draining pleural cavities.

2. Background Art

Our lungs are protected by 24 ribs, which also shape and support the chest wall. The parietal pleura is a membrane lining the chest cavity. The visceral pleura is a membrane lining the lungs. The space between these two membranes is known as the pleural space or pleural cavity. This space has a thin film of fluid to provide lubrication and cohesion between the parietal and visceral pleura. A healthy adult has approximately 20 to 25 mL of fluid in the pleural space. Normally, the drainage of this space is regulated by the lymphatic circulation.

Pleural effusion is the accumulation of pathological quantities of fluid in the pleural space. Excess pleural fluid may be caused by liver or kidney failure, congestive heart failure, infection or malignancy blocking the lymphatic system. Four types of fluids may accumulate in the pleural space: blood (hemothorax), serous fluid (hydrothorax), chyle (chylothorax), and pus (pyothorax or empyema). In addition, pneumothorax (collapsed lung) may be caused by trauma or other pathological conditions.

Each of these conditions generally requires the placement of a chest tube to drain the air or fluids placing the patient in danger. The chest tube is a flexible tube that is inserted through the side of the chest into the pleural space. It is used to remove air, fluid, or pus from the pleural space. The free end of the tube is usually attached to an underwater seal, below the level of the chest. This allows air or fluid to escape from the pleural space and prevents anything from returning to the chest. Alternatively, the tube can be attached to a flutter valve to allow patients more mobility.

Chest tubes are usually inserted under local anesthesia. The skin over the area of insertion is first cleansed with antiseptic solution before sterile drapes are placed around the area. The local anesthetic is injected into the skin and down to the muscle. After the area is numb, a passage is made through the skin and muscle into the chest. This is often carried out by performing an incision in the skin with a scalpel blade. Then, the surgeons will use a Kelly clamp to perform blunt dissection of the soft tissues. There is no depth control in the Kelly clamp which may inadvertently go too far inside the pleural cavity. Once the tube is in place it is stitched to the skin to prevent it falling out and a dressing applied to the area. The tube stays in for as long as there is air or fluid to be removed or as long as there is risk of air gathering.

The normal procedure for securing the chest tube by suturing it to the skin may be undesirable in an emergency environment. Thus, a method to secure a chest tube without the use of needles may be advantageous where time and safety is a significant factor. Such situations may include in a rescue helicopter, ambulance, or the site of an accident where resources and space may be limited. Accordingly, there exists a need for better chest tubes that is easily secure in a patient.

### SUMMARY OF INVENTION

One aspect of the invention relates to secured chest tubes. A chest tube in accordance with one embodiment of the invention includes a hollow tube having a first end and a second end; a first securing device disposed proximate the first end; and a mechanism to activate the first securing device.

Another aspect of the invention relates to methods for draining a pleural cavity with a chest tube. A method in accordance with one embodiment of the invention includes the steps of: (1) inserting a chest tube into the pleural cavity, wherein the chest tube comprising a hollow tube comprising: a first end; and a second end; and a first securing device disposed proximate the first end; and (2) activating the first securing device.

Other aspects and advantages of the invention will be apparent from the following description and the appended claims.

### BRIEF DESCRIPTION OF DRAWINGS

FIG. 1 shows a chest tube having a first securing device in accordance with one embodiment of the present invention.

FIG. 2 shows a chest tube inserted in a chest wall in accordance with one embodiment of the present invention, wherein a first securing device is activated to prevent the chest tube from falling out of the chest wall.

FIG. 3 shows a chest tube having a first and second securing devices in accordance with one embodiment of the present invention.

FIG. 4 shows a chest tube with a first and second securing devices, wherein the second securing device comprises a plurality of securing elements in accordance with one embodiment of the present invention.

FIG. 5 shows a chest tube with first and second securing devices, wherein the second securing device is slidably fitted over an outside of the chest tube, in accordance with one embodiment of the present invention.

FIG. 6 shows a flow chart illustrating a method in accordance with one embodiment of the invention.

### DETAILED DESCRIPTION

In one aspect, embodiments of the present invention relate to chest tubes used to drain fluids (gas or liquids) from pleural cavities. Chest tubes in accordance with embodiments of the invention include features that can be deployed to secure the chest tubes in the chest walls once inserted. In another aspect, embodiments of the present invention relate to methods for using chest tubes to drain fluids from pleural cavities. Methods in accordance with embodiments of the invention include deploying, after insertion of the chest tubes, the securing devices incorporated on the chest tubes. There is no need to use a needle to suture the chest tube to the chest wall. By leaving out the needles, the safety is greatly improved in an emergency situation or environment.

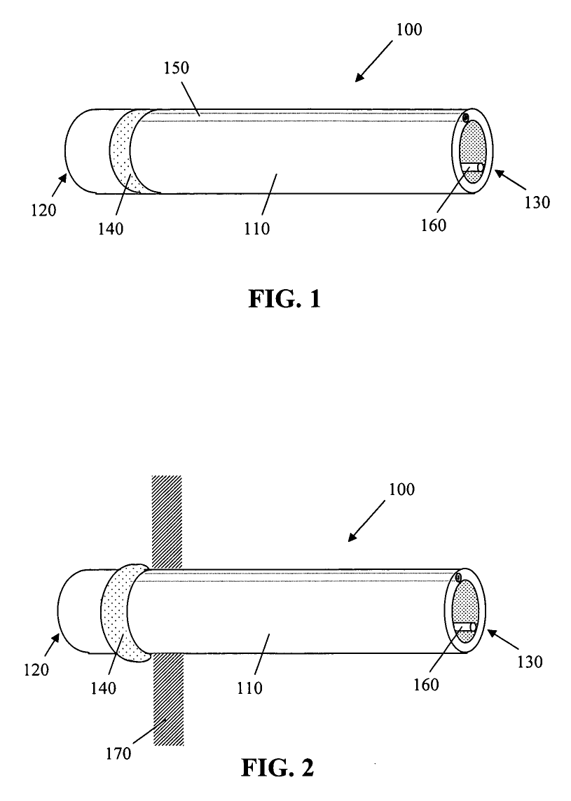

Referring to FIG. 1, in one embodiment of the present invention, a chest tube 100 comprises a hollow tube 110 having a first end 120 and a second end 130. A securing device 140 is disposed proximate the first end 120. The securing device 140, which are activated to provide a barrier so that the chest tubes will not fall off once inserted. The securing device 140 may function by various mechanisms, for example, inflatable materials or hinged flanges, and may be activated by appropriate mechanisms depending on the types of the securing device 140.

In accordance with some embodiments, securing devices (shown as 140 in FIG. 1) made of an inflatable material may be activated by a fluid (gas or liquid) introduced via conduits in the chest tubes. The conduits may be small tubes included in the lumen of the chest tubes or passages bored in the walls of the chest tubes. For example, FIG. 1 shows a passage 150 (indicated by dotted lines) bored in the wall of the chest tubes and a conduit 160 disposed in the lumen of the hollow tube 110.

FIG. 2 shows a chest tube 100 in accordance with one embodiment of the invention placed through a chest wall 170. As shown, the securing device 140 is activated to prevent the chest tube 100 from falling out of the chest wall 170. In this example, the securing device 140 is an inflatable object, such as an inflatable balloon, made of rubber or an elastomer. The inflatable object may be molded onto the chest tube, adhesively attached, or attached with other means known to one skilled in the art. Before activation (e.g., inflation), the securing device 140 has an outside dimension substantially similar or smaller than the outside diameter of the chest tube 100, as shown in FIG. 1. Upon activation (e.g., inflation), the outside dimension of the securing device 140 is larger than the outside diameter of the chest tube 100, as shown in FIG. 2. Therefore, upon activation of the securing device 140, the chest tube 100 is prevented from falling out of the chest wall. The securing device 140 may be inflated by introduction of air or other fluid (gas or liquid) via the passage 150 or conduit 160 (shown in FIG. 1). After activation the passage 150 or conduit 160 may be shut off, using a valve, a plug, or the like, to keep the securing device activated. Later one, when removal of the chest tube 100 is necessary, the securing device 140 may be deactivation (e.g., deflated).

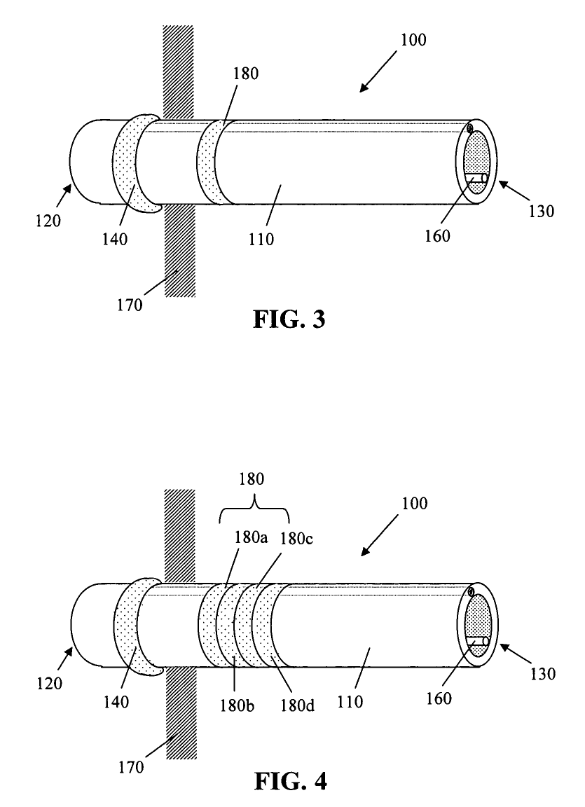

In another embodiment, shown in FIG. 3, a first securing device 140 is disposed proximate a first end 120 of a chest tube 100. Additionally, a second securing device 180 is disposed at a distance from the first securing device 140 such that the second securing device 180 is closer to a second end 130 than the first securing device 140 is. In other words, the second securing device 180 is disposed between the first securing device 140 and the second end 130. The second securing device 180 is used to secure the chest tube 100 from outside the chest wall 170. That is, when the second securing device 180 is activated, it can prevent accidental advancement of the chest tube 100 further into the pleural space. This can prevent accidental injuries to the lung.

Securing devices 140 and 180 may be made of the same or different materials, such as an elastomer or rubber as described above. Each of the first securing device 140 and the second securing device 180 may have independent passage (e.g., 150 in FIG. 1) or conduit (e.g., 160 in FIG. 1) for activation. Alternatively, they may share the same passage or conduit for activation. In the latter case, both securing devices 140 and 180 will be activated or de-activated at the same time.

It is preferred that the distance between the first securing device 140 and the second securing device 180 be similar to the thickness of the chest wall 170. Because the thickness of the chest wall 170 vary with different individuals, the distance between the first securing device 140 and the second securing device 180 may need to take into account of the potential patients. Alternatively, a second securing device 180 may comprise a plurality of deployable elements that can be selectively activated to accommodate different thicknesses of chest walls.

FIG. 4 shows one embodiment of the present invention in which the second securing device 180 comprises a plurality of securing elements 180a, 180b, etc. The plurality of securing elements 180a, 180b, etc. may be selectively activated to accommodate different chest wall thicknesses between different patients. Alternatively, all the securing elements 180a, 180b, etc. may be activated simultaneously. Any securing elements confined by the chest wall will not inflate, while those outside the chest wall 170 will. Therefore, this embodiment will always produce is nice fit according to individual chest wall thicknesses.

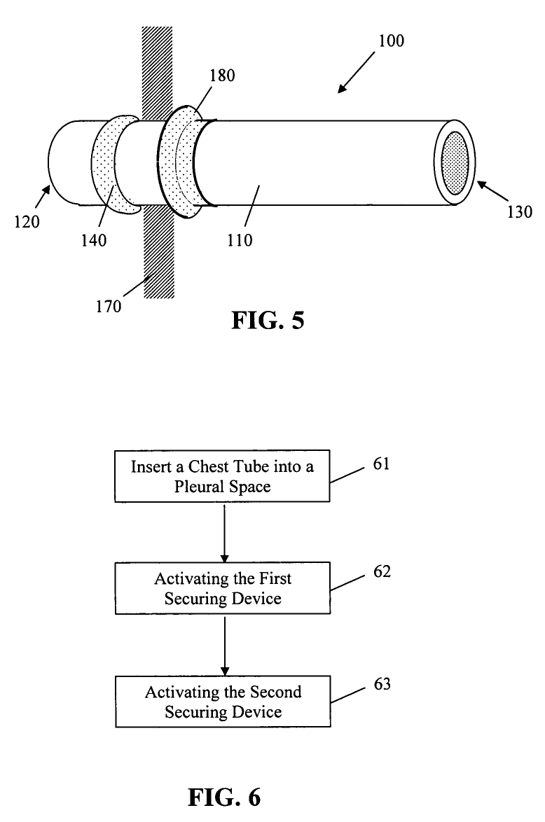

An alternate solution to the variability in chest wall thickness may be to use a second securing device 180 that is slidably fitted over the chest tube. As shown in FIG. 5, the second securing device 180 is a sleeve like structure with a flange to facilitate its attachment to the chest wall 170. In this embodiment, the slidably attached second securing device 180 can be slid along the chest tube 100 to accommodate different chest wall thicknesses. Other examples of a slidable device may include an o-ring, or any other similar structures.

In the above described embodiments, the securing devices comprise inflatable materials. In accordance with other embodiments of the invention, the securing devices need not be made of inflatable materials. For example, in some embodiments, the securing devices may comprise one or more controllable mechanical devices (e.g., flanges) disposed proximate a first end 120 of a chest tube 110. The mechanical devices may comprise a flop that can be raised and lowered. Alternatively, the mechanical devise may comprise a section of foldable membrane (e.g., an accordion like structure) that can be in the open and closed positions. When in closed positions, the flop or the foldable membrane will stay flush with the outside of the chest tube. However, when activated, the flop or the folded membrane will extend beyond the outside diameter of the chest tube to prevent the chest tube from accidentally falling out of the chest wall. The mechanical devices may be may be movable to open and closed positions via a hinge, a flexible connection, or a control line (not shown).

Some embodiments of the invention relate to chest tubes having securing devices as described above. In addition, such tech tubes may further comprise one of more conduits or passages (similar to 15 or 16 shown in FIG. 1) for delivering medications into the pleural space, while the chest tube is in place. Such a conduit or passage may be referred to as a cannula, which may be disposed in the wall of the chest tube or in the lumen of the chest tube.

Some embodiments of the invention relate to method of using the chest tubes to drain fluids from pleural space, using the chest tubes disclosed herein. As shown in FIG. 6, in accordance with one embodiment of the invention, a chest tube may be inserted into a pleural space using any techniques known in the art (step 61). After penetration of the chest wall, the first securing device of the tube may be activated to prevent the chest tube from falling out (step 62). Similarly, a second securing device may be activated to prevent the chest tube from accidentally advance further into the pleural space (step 63). Note that in some embodiments, the activation of the first and second securing devices may be simultaneously performed. One of ordinary skill in the art would recognize that a chest tube may comprise any combination of the securing elements disclosed herein.

Advantageously, embodiments of the invention provide chest tubes that can be secured after insertion without the need for suturing. Additionally, the method may be quite rapid and appropriate for use by first responders to emergencies, for example in an ambulance, rescue helicopter, or at the scene of an accident. Embodiments of the invention may allow for the chest tubes to be part of kits for routine use in first aid by trained personnel. Importantly, the securing of the chest tube may help prevent accidental removal of the tube or perhaps more seriously, accidental pushing of the chest tube further into the pleural cavity.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A chest tube, comprising:
a hollow tube comprising:
a first end; and
a second end;
a first securing device disposed proximate the first end; and
a mechanism to activate the first securing device.

2. The chest tube of claim 1, further comprising a second securing device disposed on the chest tube at a distance from the first securing device such that the second securing device is between the first securing device and the second end.

3. The chest tube of claim 1, wherein the first securing device is made of an inflatable material.

4. The chest tube of claim 2, wherein the second securing device is made of an inflatable material.

5. The chest tube of claim 2, wherein the second securing device is slidably fitted on an outside of the chest tube.

6. The chest tube of claim 1, further comprising a cannula disposed in the chest tube for delivering a medicament.

7. The chest tube of claim 6, wherein the cannula comprises a channel bored in the wall of the chest tube.

8. A method for draining a pleural cavity with a chest tube, comprising:
inserting a chest tube into the pleural cavity, the chest tube comprising:
a hollow tube comprising:
a first end; and
a second end; and
a first securing device disposed proximate the first end; and
activating the first securing device.

9. The method of claim 8, wherein the chest tube further comprises a second securing device disposed on the chest tube at a distance from the first securing device such that the second securing device is between the first securing device and the second end, and the method further comprises activating the second securing device.

10. The method of claim 9, wherein the second securing device comprises a plurality of securing elements, and the activating the second securing device comprising selectively activating one of the plurality of securing elements.

11. The method of claim 9, wherein the second securing device is slidably fitted over an outside of the chest tube, and the activating the second securing device comprises sliding the second securing device to accommodate a thickness of a chest wall.
